(19) United States
(12) Patent Application Publication
(10) Pub. No.: US 2008/0221532 A1
(43) Pub. Date: Sep. 11, 2008

(54) MICRONEEDLE SHEET AND METHOD FOR MANUFACTURING THE SAME

(75) Inventor: Shotaro Ogawa, Fujinomiya-shi (JP)

Correspondence Address: BIRCH STEWART KOLASCH & BIRCH, PO BOX 747, FALLS CHURCH, VA, 22040-0747, US

(73) Assignee: FUJIFILM Corporation, Tokyo (JP)

(21) Appl. No.: 11/951,938

(22) Filed: Dec. 6, 2007

(30) Foreign Application Priority Data
Dec. 7, 2006 (JP) 2006-330698

(51) Int. Cl. A61M 5/32

(52) U.S. Cl. 604/272

(57) ABSTRACT

In a microneedle sheet having a microneedle array composed of a large number of microneedles Formed on the surface of the sheet, the ridge lines or conical plains of the microneedles are curved inward into said microneedles. Since the ridge lines or conical planes of the microneedles have a shape curved inward into the microneedles, a microneedle sheet that can be smoothly inserted into the skin can be provided.

### BACKGROUND OF THE INVENTION

1. Field of the Invention

The present invention relates to a microneedle sheet and a method for manufacturing and thereof; and specifically to a microneedle sheet that can easily, safely and efficiently inject medicals or the like into a surface or horny layer of a skin, and a method for manufacturing such a microneedle sheet.

2. Description of the Related Art

Heretofore, a majority of methods to administer medicals or the like through the surface of a living body, such as the skin and mucous membrane, has been a method to adhere mainly a liquid or powdered substance. However, since the region to adhere these substances has been limited to the surface of the skin, the adhered medicals or the like have been removed due to sweating or the contact of foreign matters at times, and the administration of an adequate quantity has been difficult. In order to medicate medicals deep into the skin, since it has been difficult to accurately control the depth of penetration by the method using the osmosis by the diffusion of such medicals, it has been difficult to obtain satisfactory medicinal benefits.

Therefore, methods for injecting medicals by using a functional micro pile or the like described in National Publication of International Patent Application No. 2002-517300, Japanese Patent Application Laid-Open No. 2003-238347 and Japanese Patent Application Laid-Open No. 2006-51361 and inserting the tips thereof into the skin have been disclosed.

However, in the invention disclosed in National Publication of International Patent Application No. 2002-517300, since a substrate surface composed of Si, metals or the like is directly subjected to etching process when a functional micro pile is manufactured, there has been a problem of poor productivity and high costs. Since the inventions disclosed in Japanese Patent Application Laid-Open No. 2003-238347 and Japanese Patent Application Laid-Open No. 2006-51361 are methods for fabricating what is to be a functional micro pile by the injection molding of a resin material, it has been difficult to release a functional micro pile having a high aspect ratio from the mold, and to obtain a perfect product because defects arc easily produced in the edge portions, and there has been a problem of low yield in manufacture.

Also since the functional micro pile and the like manufactured by the methods described in National Publication of International Patent Application No. 2002-517300, Japanese Patent Application Laid-Open No. 2003-238347 and Japanese Patent Application Laid-Open No. 2006-51361 are manufactured not considering easy insertion into the skin, there has been a problem of difficulty in smooth insertion into the skin.

The present invention has been made in view of the above-described situations, and it is an object of the present invention to provide a microneedle sheet having the array of microneedles that can be easily inserted into a skin and can be manufactured at low manufacturing costs and at high yield, and a method for the manufacture thereof.

### SUMMARY OF THE INVENTION

The first aspect of the present invention provides a microneedle sheet comprising a microneedle array composed of a large number of pyramidal microneedles formed on the surface of the sheet, wherein the ridge lines of the pyramidal microneedles have a shape curved inward into the microneedles.

According to the first aspect of the present invention, since the ridge lines of the pyramidal microneedles have a shape curved inward into the microneedles, a microneedle sheet that can be smoothly inserted into the skin can be provided.

The second aspect of the present invention is characterized in that the maximum depth Z of the curvature of the ridge lines in the first aspect is not less than 0.04×L and not more than 0.2×L when the length of the segment of a lice connecting the starting point and the ending point of tire ridge lines is denoted by L.

According to the second aspect of the present invention, it is preferable that the maximum depth Z of the curvature of the ridge lines in the first aspect is not less than 0.04×L and not more than 0.2×L when the length of the segment of a line connecting the starting point to the ending point of the ridge lines is denoted by L. In aspects of the present invention, the starting point and the ending point of the ridge lines are defined so that when a line parallel to the bottom surface is drawn at the location of half the height h of the tip A from the bottom surface of a pyramid, h/2, and the point where the parallel line intersects the ridge line is M, and a point B where the segment AM equals to the segment BM is taken, the tip A is the starting point of the ridge line, and the point B is the ending point of the generating line of the ridge line.

The third aspect of the present invention is characterized in that the ridge lines of the pyramidal microneedles in the first or second aspect of the present invention are protruded from the pyramidal planes between the ridge lines.

According to the third aspect of the present invention, since the ridge lines of the pyramidal microneedles are protruded than the pyramidal planes between the ridge lines, a microneedle sheet that can be more smoothly inserted into the skin can be provided.

The fourth aspect of the present invention is characterized in that the radius of curvature showing the sharpness of the ridge lines of the pyramidal microneedles in any one of the first to third aspects of the present invention is not more than 10 μm.

According to the fourth aspect of the present invention, the radius of curvature of the ridge lines is preferably not more than 10 μm.

The firth aspect of the present invention provides a microneedle sheet comprising a microneedle array having a large number of conical microneedles formed on the surface of the sheet, wherein the conical planes of the conical microneedles are curved inward into the microneedles.

According to the fifth aspect of the present invention, since the conical planes of the conical microneedles are curved inward into the microneedles, a microneedle sheet that can be smoothly inserted into the skin can be provided.

The sixth aspect of the present invention is characterized in that the maximum depth Z′ of the curvature of the conical planes in the fifth aspect is not less than 0.04×L′ and not more than 0.2×L′ when the length of the segment of a line connecting the starting point and the ending point of the generating lines of the conical planes is denoted by L′.

According to the sixth aspect of the present invention, the maximum depth Z′ of the curvature of the conical planes is preferably not less than 0.04×L′ and not more than 0.2×L′ when the length of the segment of a line connecting the starting point and the ending point of the generating lines of the conical planes is denoted by L′. In aspects of the present invention, the starting point and the ending point of the conical planes are defined so mat when a line parallel to the bottom surface is drawn at the location of half the height h of the tip A from the bottom surface of a cone, h/2, and the point where the parallel line intersects the conical plane is M, and a point B where the segment AM equals to the segment BM is taken, the tip A is the starting point of the generating line of the conical plane, and the point B is the ending point of the generating line of the conical plane.

The seventh aspect of the present invention is characterized in that the radius of curvature showing the sharpness of the tips of the pyramidal microneedles in any one of the first to sixth aspects is not more than 5 μm.

According to the seventh aspect of the present invention, since the radius of curvature of the pyramidal microneedles is not more than 5 μm, a microneedle sheet can be more smoothly inserted into the skin.

The eighth aspect of the present invention is characterized in that the sides or diameter of the bottom surfaces of the microneedles is not less than 30 μm and not more than 300 μm and the height thereof is not less than 50 μm and not more than 1000 μm in any one of the first to seventh aspects.

According to the eighth aspect of the present invention, the sides (in the case where the microneedles are pyramidal) or diameter (in the case where the microneedles are conical) of the bottom surface of the microneedle is preferably not less than 30 μm and not more than 300 μm and the height thereof is preferably not less than 50 μm and not more than 1000 μm.

The ninth aspect of the present invention provides a method for manufacturing a microneedle sheet having a microneedle array composed of a large number of pyramidal or conical microneedles formed on the surface of the sheet, comprising: polymer solution applying step for applying a polymer solution prepared by dissolving a polymer resin that can be gelled in a solvent onto a stamper for forming the microneedle array; a polymer solution drying step for gelling the applied polymer solution to form a solidified body whose volume is shrunk at least at a certain shrinkage factor, and evaporating the solvent to dry the gelled solidified body, and a peeling step for peeling the dried solidified body from the stamper.

According to the ninth aspect of the present invention, in a method for manufacturing a microneedle sheet having a microneedle array composed of a large number of pyramidal or conical microneedles formed on the surface of the sheet, a microneedle sheet having a microneedle array composed of a large number of pyramidal or conical microneedles of a shape whose the ridge lines are curved inward into the microneedles can be manufactured by a polymer solution applying step for applying a polymer solution prepared by dissolving a polymer resin that can he gelled in a solvent onto a stamper for forming the microneedle array; a polymer solution drying step for gelling the applied polymer solution to form a solidified body whose volume is shrunk at least at a certain shrinkage factor, and evaporating the solvent to dry the gelled solidified body, and a peeling step for peeling the dried solidified body from the stamper. Therefore, a microneedle sheet that can he smoothly inserted into the skin can be manufactured. In addition, since the microneedle sheet is manufactured using such steps, microneedle sheets can be provided at low manufacturing costs and high yield.

The tenth aspect of the present invention is characterized in that the certain shrinkage factor in the ninth aspect is at least 70%.

According to the tenth aspect of the present invention, since the certain shrinkage factor is at least 70%, a microneedle sheet that can be more smoothly inserted into the skin can be manufactured.

The eleventh aspect of the present invention is characterized in that at least one of the selection of the kind of the polymer resin, the adjustment of the concentration of the polymer resin in the solvent, the addition of a gelling agent, and the adjustment of drying conditions is performed in the ninth or tenth aspect, so that the shrinkage factor of the solidified body becomes at least 70%.

According to the eleventh aspect of the present invention, by performing at least one of the selection of the kind of the polymer resin, the adjustment of the concentration of the polymer resin in the solvent, the addition of a gelling agent, and the adjustment of drying conditions, the shrinkage factor of the solidified body can be at least 70%.

The twelfth aspect of the present invention is characterized in that the polymer resin in any one of the ninth to eleventh aspects is water soluble.

According to the twelfth aspect of the present invention, since the polymer resin is water soluble, the manufactured microneedle sheet can be safely inserted into the skin.

The thirteenth aspect of the present invention is characterized in that the polymer resin in any one of the ninth to twelfth aspects is one selected from a group consisting of gelatin, agarose, maltose, pectin, gellan gum, carrageenan, xatrthan gum, alginic acid, starch, and the combination of these materials.

According to the thirteenth aspect of the present invention, by the use of such materials, a microneedle sheet having a shape wherein the ridge lines of microneedles are preferably curved inward into the microneedles can be manufactured.

The fourteenth aspect of the present invention is characterized in that when the polymer resin in any one of the ninth to thirteenth aspects is peeled off from the stamper, a backing sheet is adhered to the stamper via the polymer resin, and the backing sheet is peeled off from the stamper.

According to the fourteenth aspect of the present invention, since a backing sheet is adhered to the stamper via the polymer resin, and the polymer resin is peeled off from the stamper, a durable microneedle sheet can be manufactured at high yield.

According to the aspects of the present invention as have been described, a microneedle sheet having the array of microneedles that can be smoothly inserted into the skin without damaging the shapes of the formed microneedles, and can be manufactured at low manufacturing costs and high yield; and a method for manufacturing such a microneedle sheet can be provided.

### BRIEF DESCRIPTION OF THE DRAWINGS

FIG. 1 is a flow chart of a method for manufacturing a microneedle sheet according to an embodiment of the present invention;

FIG. 2 is a configuration diagram showing a system for manufacturing a microneedle sheet according to an embodiment of the present invention;

FIGS. 3A, 3B and 3C are process diagrams (1) showing a method for manufacturing a microneedle sheet according to an embodiment of the present invention;

FIGS. 4A, 4B and 4C arc process diagrams (2) showing a method for manufacturing a microneedle sheet according to an embodiment of the present invention;

FIG. 5A is a perspective view of a pyramidal microneedle of a microneedle sheet according to an embodiment of the present invention;

FIG. 5B is a sectional view of a pyramidal microneedle of a microneedle sheet according to an embodiment of the present invention;

FIG. 6A is a perspective view of a conical microneedle of a microneedle sheet according to an embodiment of the present invention;

FIG. 6B is a sectional view of a conical microneedle of a microneedle sheet according to an embodiment of the present invention;

FIG. 7 is an illustrative diagram for illustrating the starting point and the ending point of the ridge line or the generating line of the conical surface in an embodiment of the present invention; and

### DETAILED DESCRIPTION OF THE PREFERRED EMBODIMENTS

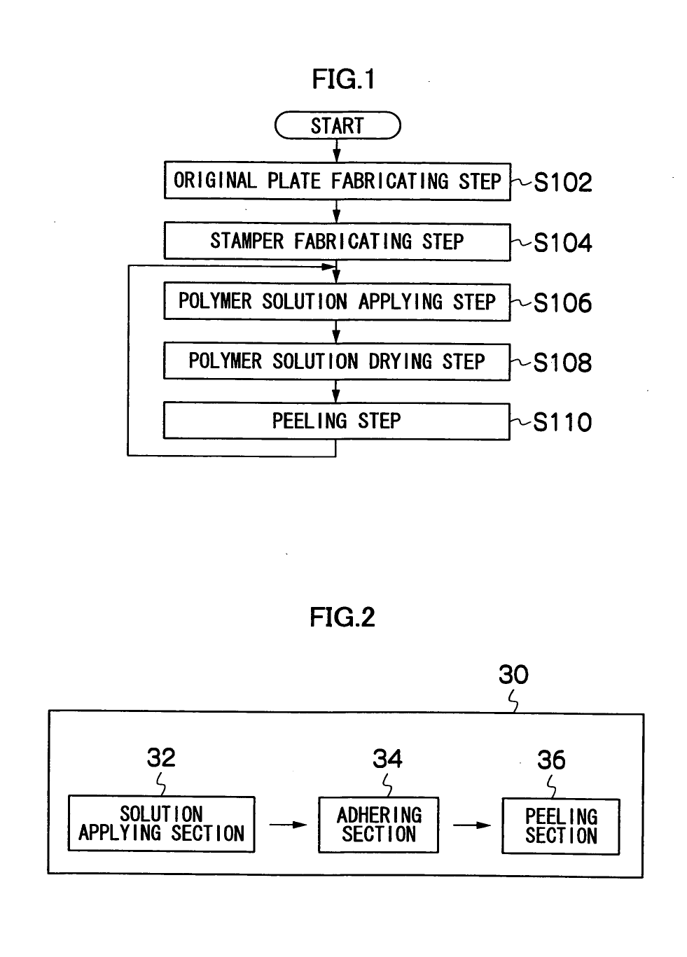

A microneedle sheet and a method for the manufacture thereof according to embodiments of the present invention will be described below. FIG. 1 shows a flow chart of an embodiment of the present embodiment; and FIG. 2 is a configuration diagram showing an example of a system for manufacturing a microneedle sheet.

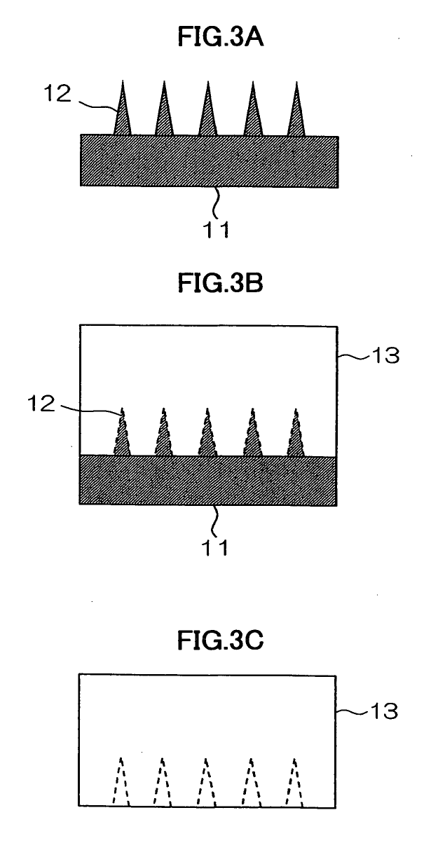

First, Step 102 (S102) for fabricating an original plate shown in FIG. 1 is carried out. Specifically, as shown in FIG. 3A, an original plate for fabricating a stamper for the manufacture of a microneedle sheet is fabricated.

There are two kinds of methods for fabricating the original plate 11. The first method is a method wherein a photo-resist is applied onto a Si substrate, exposed, developed, and subjected to etching such as RIE (reactive ion etching) to fabricate the array of conical portions 12 on the surface of the original plate 11. When etching such as RIE is performed, a conical shape can be formed by performing etching from a diagonal direction while rotating the Si substrate.

The second method is a method to form the array of pyramidal or other portions 12 on the surface of the original plate 11 on a metal substrate such as Ni using a cutting tool such as a diamond bit.

Next, Step 104 (S104) for fabricating a stamper shown in FIG. 1 is carried out. Specifically, as shown in FIG. 3B, a stamper 13 is fabricated from the original plate 11. Although a method using Ni electrocasting or the like is used for the fabrication of an ordinary stamper 13, since the original plate 11 has a conical or pyramidal shapes with sharp tips, in the present embodiment, three methods enabling manufacture at low costs, so that the shapes can be accurately transferred to and released from the stamper 13, are considered.

The first method is a method wherein a silicone resin composed of PDMS (polydimethyl siloxane, for example, Silguard 184 manufactured by Dow Corning Co. Ltd) to which a hardening agent is added is poured into the original plate 11, cured by heat treatment at 100° C., and released from the original plate 11. The second method is a method wherein a UV curing resin that is curable by ultraviolet irradiation is poured into the original plate 11, irradiated by ultraviolet beams in a nitrogen atmosphere, and released from the original plate 11. The third method is a method wherein a plastic resin, such as polystyrene and PMMA (polymethyl methacrylate) dissolved in an organic solvent is poured into the original plate 11 that has a parting agent applied thereon, dried to evaporate the organic solvent to be cured, and released from the original plate 11.

The stamper 13 thus fabricated is shown in FIG. 3C. The stamper 13 can be easily replicated any number of times using any one of the above-described three methods.

Next, Step 106 (S106) for applying a polymer solution shown in FIG. 1 is carried out. Specifically, a solution wherein a polymer resin is dissolved is applied onto the surface having a concave-convex pattern corresponding to the microneedles of the stamper 13 fabricated in the stamper fabricating step. In the solution, an appropriate quantity of medicals to be administered can be mixed.

The examples of specific methods for applying a solution wherein a polymer resin is dissolved include an applying method using a spin coater. Since the case where the solution containing the polymer resin cannot enter into the bottom of the dents for forming microneedles formed in the stamper 13 due to the presence of the air is considered, it is desirable to carry out this step in a state of reduced pressure. The Step 106 for applying a polymer solution is carried out in the solution applying section 32 of the microneedle sheet manufacturing system 30 shown in FIG. 2.

For inserting the microneedles into the surface of the skin to a depth o f several hundred micrometers, it is required that (1) the tips of the microneedles are sufficiently sharp and the diameters of the microneedles inserted into the skin are sufficiently thin (the length/diameter aspect ratio is high); and (2) the microneedles have a sufficient strength (the microneedles do not bend).

Although a thin and sharp shape is required for (1), this conflicts with (2). If the microneedles are excessively thin, they are bent at the tips or the roots, and if they are excessively thick, they cannot be inserted. As a method to improve the confliction, it can he considered that a material as hard as possible is used, and the ridge lines of the conical or pyramidal bodies are curved inwards to make the roots of the bodies widened and difficult to break while the bodies have sufficiently sharp tips.

Further as the process for inserting the microneedles into the skin, it is required that (3) the pores made by insertion are spread with the deepening of insertion after the tips of the microneedles have been inserted into the skin. For this purpose, the tips of the microneedles having a pyramidal shape, which have sharp edges of the ridge line, are more preferable than a conical shape.

It is not easy to process the stamper 13 having such a shape. However, even if the stamper 13 is conical or pyramidal, the above-described (1) to (3) can be satisfied if the material significantly shrinks during the formation of the stamper 13. According to embodiments of the present invention, by injecting such a material having a large shrinkage factor into the stamper 13, making the material gelled, drying the gelled material, and allowing the material to shrink in the mold. Thus, the needle sheet that satisfies the above-described (1) to (3) can be realized.

The polymer resin used for applying is preferably water-soluble, and a peolymer resin formed by dissolving the powder of gelatin, agarose, maltose, pectin, gellan gum, carrageenan, xanthan gum, alginic acid, starch or the like in warm water is particularly preferable. Although the concentration of the polymer resin depends on the material, about 20% is preferable. The solvent used for dissolving the polymer resin is not limited to warm water, but can be any solvent as long as it is volatile. For example, alcohols and the like can also be used. Medicals can be added after dissolving the polymer resin. If the solution is cooled to, for example, 30 to 60° C., the medicals having low heat resistance can also be added.

Next, Step 108 (S108) for drying a polymer solution shown in FIG. 1 is carried out. Specifically, the applied solution wherein the polymer resin is dissolved is dried by blasting warm air.

In one method, cool air is blasted at 10 to 15° C. first to gel the surface, and warm air is blasted at 10 to 20 m/s. The warm air is preferably dehumidified, and for example, the temperature is 40 to 60° C. and the relative humidity is not more than 15%, preferably, not more than 10%.

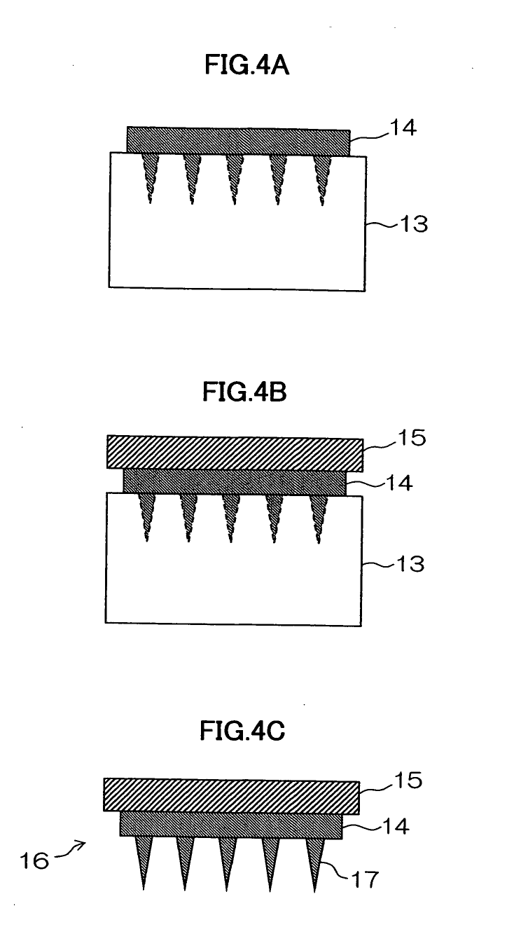

Alternatively, the applied solution wherein the polymer resin is dissolved can be gelled by flowing cool air of low humidity. In this case, cool air of 10 to 15° C. is blasted for a longer time than the above-described case, and thereafter warm air is blasted as described above. In this case, when warm air of a high temperature is flowed for drying, if the temperature of the warm air is excessively high, the gelation of the solution wherein the polymer resin is dissolved may be returned or the efficacy of some medicals may be changed by decomposition or the like due to heating. Therefore, care should be taken for the temperature of the warm air to be blasted. As described, by drying the applied solution wherein the polymer resin is dissolved, or by drying the polymer solution after gelation of the polymer solution, the polymer solution is solidified to form a polymer resin 14 as shown in FIG. 4A.

At this time, by gelling the polymer solution and solidifying the polymer resin 14, the polymer resin is more significantly shrunk than the state when the solution wherein the polymer resin is dissolved is applied. Thereby, the microneedle 17 has a shape whose ridge lines 17A, 17A . . . are curved inward into the microneedle as shown in 5A.

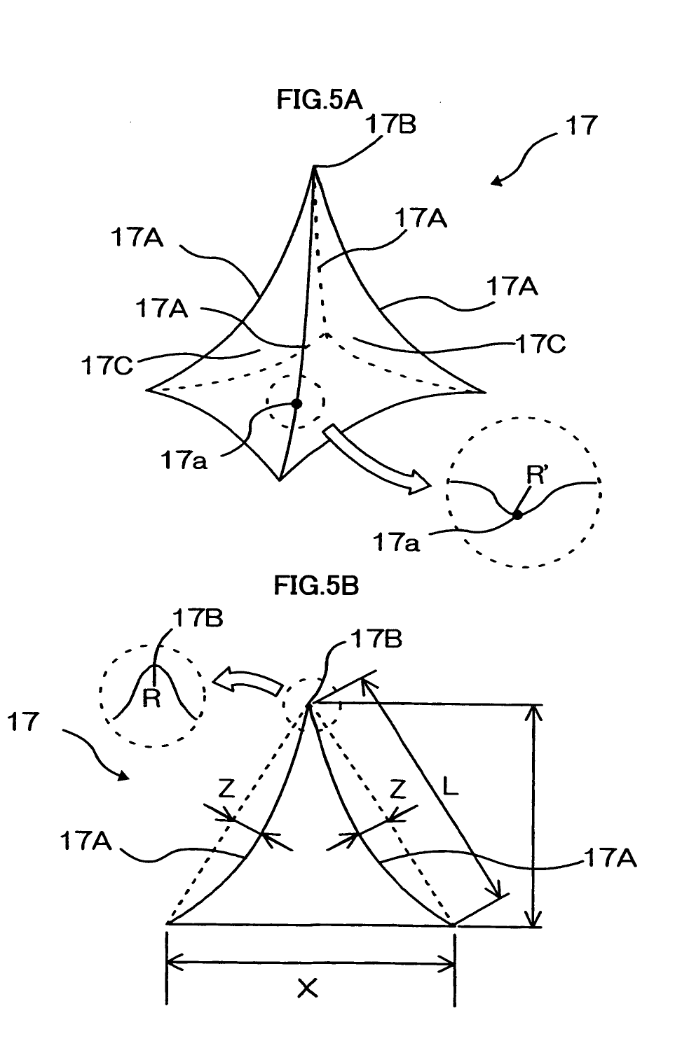

FIG. 5B is a diagram of a microneedle 17 viewed from the side. Here, it is preferable that the ridge lines 17A of the pyramidal microneedle are protruded than the pyramidal planes 17C between the ridge lines. A side X of a bottom surface of the microneedle 17 is preferably not less than 30 μm and not more than 300 μm, and the height Y thereof is preferably not less than 50 μm and not more than 1000 μm. When the length of a line segment connecting the starting point and the ending point of a ridge line is L, the maximum depth Z of the curvature of a ridge line 17A is preferably not less than 0.04×L and not more than 0.2×L. The radius of curvature R′, which shows the sharpness of the ridge line 17A of a microneedle, is preferably not more than 10 μm (refer to the cross section at the point 17a on the ridge line 17A shown in FIG. 5A), and the radius of curvature R, which shows the sharpness of the tip 17B of a microneedle, is preferably not more than 5 μm (refer to FIG. 5B).

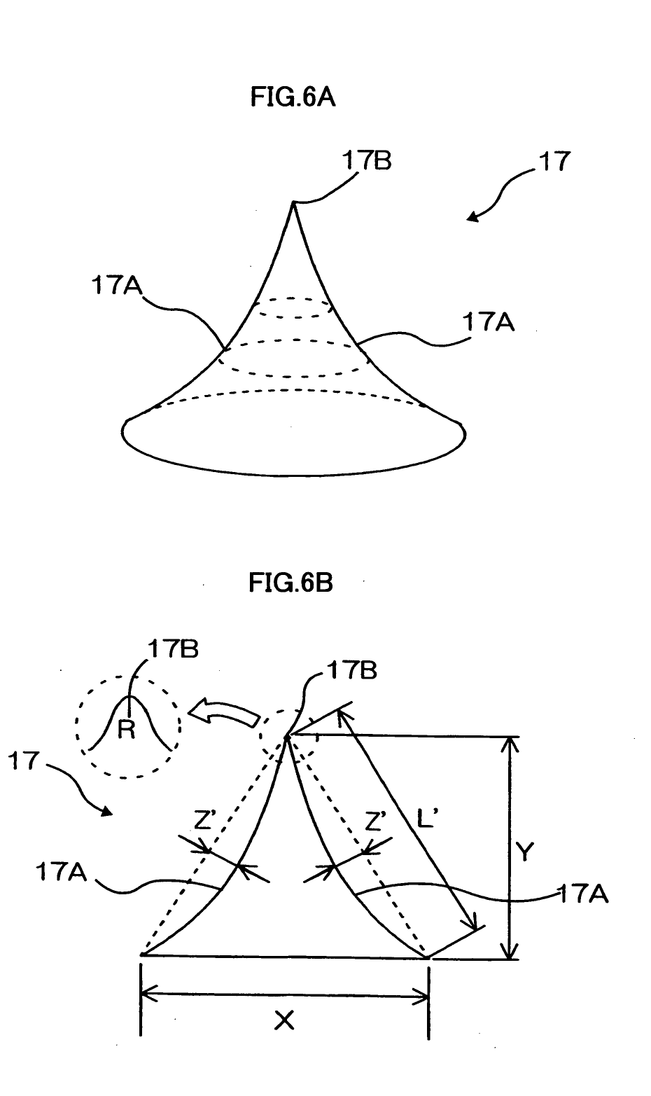

Although a four-sided pyramidal microneedle 17 is shown in FIG. 5, a conical microneedle as shown in FIG. 6 and other pyramidal microneedles can also be manufactured in the same manner. In a conical microneedle, the maximum depth Z′ of the curvature of the conical planes is preferably not less than 0.04×L′ and not more than 0.2×L′ when the length of the segment of a line connecting the starting point to the ending point of the generating lines of the conical planes is denoted by L′.

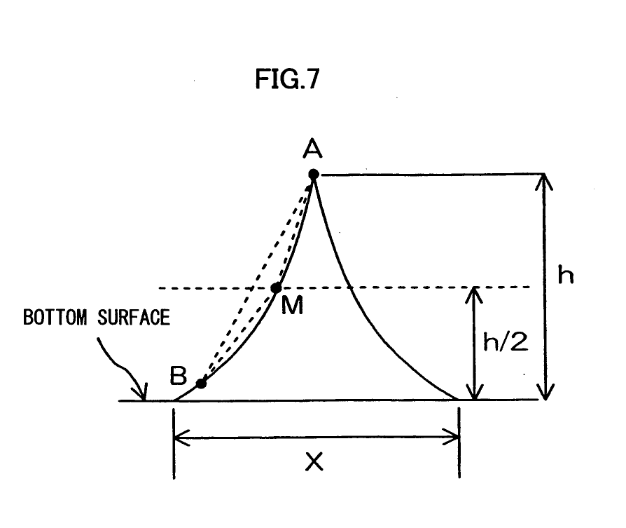

In an embodiment of the present invention, the starting point and the ending point of the ridge lines or the generating lines of the conical planes are defined as follows. As shown in FIG. 7, a line parallel to the bottom surface is drawn at the location of half the height h of the tip A from the bottom surface of a pyramid or a cone, h/2, and the point where the parallel line intersects the ridge line or the conical plane is M, and a point B where the segment AM equals to the segment BM is taken on the ridge line or the conical plane. At this time, the tip A (point A) is defined as the starting point of the ridge line or the generating line of the conical plane, and the point B is defined as the ending point of the ridge line or the generating line of the conical plane.

Next, Step 110 (S110) for peeling shown in FIG. 1 is carried out. Specifically, as shown in FIG. 4B, after adhering a PET (polyethylene terephthalate) sheet 15, which is a backing sheet on which an adhering layer is formed, onto a dried and cured polymer resin 14 on the stamper 13 in the preceding step for drying polymer solution, the PET sheet 15 is peeled. Thereby, a microneedle sheet 16 is completed as shown in FIG. 4C. The adhesion of the PET sheet 15 in the peeling step S110 is performed in the adhering section 34 in the microneedle sheet manufacturing system 30 shown in FIG. 2, and the peeling of the solidified polymer resin 14 together with the adhered PET sheet 15 is performed in the peeling section 36 in the microneedle sheet manufacturing system 30 shown in FIG. 2

Normally as in the present embodiment, when the structure composed of microneedles having a high aspect ratio is peeled off the stamper 13, since the contact area is large, a strong stress is applied, and microneedles are broken and are left in the stamper 13 without being peeled off, resulting in fatal defects in the fabricated microneedle sheet 16. Based on this, the stamper 13 is preferably composed of materials that can be easily peeled off. By using a highly elastic and flexible material 30 for constituting the stamper 13, stress applied to the microneedles during peeling can be relieved. Furthermore, in the peeling step, by peeling as flipping it from the end portion using a roller 18 as shown in FIG. 8, stress can be further relieved. From the above point of view, as the material to constitute the stamper 13 a material which easily undergoes elastic deformation, such as silicone rubber is preferable.

To evaporate moisture remaining on the microneedles 17 on the surface of the polymer resin 14, dried air may be blasted again after peeling. Specifically, it is preferable to package the microneedle sheet after reducing the moisture content hi the polymer resin immediately before packaging to not more than 10%, more desirably not more than 5%.

Since the stamper 13 can be used for a plurality of times, using the stamper 13 after the peeling step of Step 110, by repeating tire polymer solution applying step of Step 106, the polymer solution drying step of Step 108, and the peeling step of Step 110, a plurality of microneedle sheets 16 can be fabricated in a short time at low manufacturing costs. In addition, since the stamper 13 cannot be permanently used, when the stamper 13 becomes no longer usable, it can be fabricated by carrying out the stamper fabricating step of Step 104.

As described above, the method for manufacturing a microneedle sheet having a microneedle array composed of a large number of pyramidal or conical microneedles formed on the surface of the sheet includes: a polymer solution applying step for applying a polymer solution prepared by dissolving a polymer resin that can be gelled in a solvent onto a stamper for forming the microneedle array; a polymer solution drying step for gelling the applied polymer solution to form a solidified body whose volume is shrunk at least at a certain shrinkage factor, and evaporating the solvent to dry the gelled solidified body, and a peeling step for peeling the dried solidified body from the stamper, a microneedle sheet that can be smoothly inserted into the skin can be manufactured. Thereby, a microneedle sheet 16 having a microneedle array composed of a large number of pyramidal or conical microneedles of a shape whose the ridge lines 17A are curved inward into the microneedles 17 can be manufactured. In addition, since the microneedle sheet 16 is manufactured using such steps, microneedle sheets 16 can be provided at low manufacturing costs and high yield.

Here, the shrinkage factor is preferably at least 70%. The shrinkage factor of the solidified body of at least 70% can be achieved by performing at least one of the selection of the kind of the polymer resin, the adjustment of the concentration of the polymer resin in the solvent, the addition of a gelling agent, and the adjustment of drying conditions.

In the practical manufacture of microneedle sheets 16, by preparing a plurality of stampers 13 and simultaneously using the stampers 13, microneedle sheets 16 can be manufactured at a high productivity.

### EXAMPLES

The examples of the present embodiment will be described.

### Example 1

In example 1, a metal plate composed of Ni was cut using a diamond tool to fabricate an original plate 11 wherein an array of four-sided pyramids each having a base X of a pyramidal portion 12 shown in FIG. 5B of 200 μm, a height Y of 400 μm, and a pitch of 1000 μm.

A silicone resin (PDMS) was poured into the original plate 11 and cured to fabricate a stamper 13 of an inverted shape of the original plate 11 shown in FIG. 3C.

After gelatin was dissolved in water, stirred and swollen, the solution was heated to 40° C. and dissolved to prepare a solution wherein a polymer resin of a gelatin concentration of 20% was dissolved. The solution was applied onto the concave-convex surface of the stamper 13 using a spin coater. An appropriate quantity of medicals to be administered is mixed in the solution.

After blasting cool air of a temperature of 15° C. to the coating of the solution wherein the polymer resin was dissolved prepared by dissolving gelatin in water for 20 seconds to complete gelling, warm air of a temperature of 50° C. and a relative humidity of 15% was blasted for 30 minutes to sufficiently dry and solidify the coating.

Thereafter, as shown in FIG. 4B, a PET sheet having a thickness of 120 μm on which an adhering layer is formed is placed on and adhered to the solidified polymer resin 14 on the stamper 13, and as shown in FIG. 8, the solidified polymer resin 14 is peeled as flipping it from the end portion using a roller 18. Thereby, a highly accurate microneedle sheet 16 that can be smoothly inserted into the skin can be fabricated.

### Example 2

The concentration of gelatin and drying conditions in example 1 were changed to change the shrinkage factor of gelatin when solidified, and a microneedle sheet 16 was fabricated. Samples having shrinkage factors from 50% to 75% measured from observation through a microscope were prepared. A constant load per needle was given to these samples to check whether the needles can be inserted into a simulated skin (silicone rubber) or not. The results are shown in Table 1. As Table 1 shows, it was confirmed that as the shrinkage was larger, the side planes (ridge lines) of a pyramid were more curved inward, and the needles could be more easily inserted at a low load.

TABLE 1

| Shrinkage factor | Load required for insertion |
|---|---|
| 75% | Inserted at 10 g/needle or more |
| 70% | Inserted at 10 g/needle or more |
| 60% | Inserted at 30 g/needle or more |
| 50% | Inserted at 40 g/needle or more |

A microneedle sheet and a method for the manufacture thereof according to the embodiments of the present invention have been described in detail; however, the present invention is not limited to the described examples, but various improvements and modifications can be made within the range not deviated from the scope of the present invention.

What is claimed is:

1. A microneedle sheet comprising a microneedle array composed of a large number of pyramidal microneedles formed on the surface of the sheet, wherein
ridge lines of said pyramidal microneedles have a shape curved inward into said microneedles.

2. The microneedle sheet according to claim 1 wherein the ridge lines of said pyramidal microneedles are protruded than pyramidal planes between said ridge lines.

3. The microneedle sheet according to claim 1 wherein the radius of curvature showing the sharpness of tips of said pyramidal microneedles is not more than 5 μm.

4. The microneedle sheet according to claim 1 wherein the sides or diameter of the bottom surfaces of said microneedles is not less than 30 μm and not more than 300 μm and the height thereof is not less than 50 μm and not more than 1000 μm

5. The microneedle sheet according to claim 1 wherein the maximum depth Z of the curvature of said ridge lines is not less than 0.04×L and not more than 0.2×L when the length of a segment of a line connecting a starting point and a ending point of the ridge lines is denoted by L.

6. The microneedle sheet according to claim 5 wherein the ridge lines of said pyramidal microneedles are protruded than pyramidal planes between said ridge lines.

7. The microneedle sheet according to claim 1 wherein the radius of curvature showing the sharpness of the ridge lines of the pyramidal microneedles is not more than 10 μm.

8. A microneedle sheet comprising a microneedle array having a large number of conical microneedles formed on the surface of the sheet, wherein
conical planes of said conical microneedles arc curved inward into the microneedles.

9. The microneedle sheet according to claim 8 wherein the maximum depth Z′ of curvature of said conical planes is not less than 0.04×L′ and not more than 0.2×L′ when the length of a segment of a line connecting a starting point and a ending point of generating lines of the conical planes is denoted by L′.

10. The microneedle sheet according to claim 8 wherein the radius of curvature showing the sharpness of tips of said pyramidal microneedles is not more than 5 μm.

11. The microneedle sheet according to claim 8 wherein the sides or diameter of the bottom surface of said microneedle is not less than 30 μm and not more than 300 μm and the height thereof is not less than 50 μm and not more than 1000 μm

12. A method for manufacturing a microneedle sheet having a microneedle array composed of a large number of pyramidal or conical microneedles formed on the surface of the sheet, comprising:
a polymer solution applying step for applying a polymer solution prepared by dissolving a polymer resin that can be gelled in a solvent onto a stamper for forming said microneedle array;
a polymer solution drying step for gelling said applied polymer solution to form a solidified body whose volume is shrunk at least at a certain shrinkage factor, and evaporating said solvent to dry the gelled solidified body, and
a peeling step For peeling said dried solidified body from said stamper.

13. The method for manufacturing a microneedle sheet according to claim 12, wherein at least one of selection of the kind of said polymer resin, adjustment of concentration of said polymer resin in said solvent, addition of a gelling agent, and adjustment of drying conditions is performed, so that the shrinkage factor of said solidified body becomes at least 70%.

14. The method for manufacturing a microneedle sheet according to claim 12, wherein said polymer resin is water soluble.

15. The method for manufacturing a microneedle sheet according to claim 12, wherein said certain shrinkage factor is at least 70%.

16. The method for manufacturing a microneedle sheet according to claim 15, wherein said polymer resin is water soluble.

17. The method for manufacturing a microneedle sheet according to claim 15, wherein at least one of selection of the kind of said polymer resin, adjustment of concentration of said polymer resin in said solvent, addition of a gelling agent, and adjustment of drying conditions is performed, so that the shrinkage factor of said solidified body becomes at least 70%.

18. The method for manufacturing a microneedle sheet according to claim 17, wherein said polymer resin is water soluble.

19. The method for manufacturing a microneedle sheet according to claim 12, wherein said polymer resin is one selected from a group consisting of gelatin, agarose, maltose, pectin, gellan gum, carrageenan, xanthan gum, alginic acid, starch, and the combination of the materials.

20. The method for manufacturing a microneedle sheet according to claim 12, wherein when said polymer resin is peeled off from said stamper, a backing sheet is adhered to said stamper via said polymer resin, and said backing sheet is peeled off from said stamper.
